(19) United States
(12) Patent Application Publication
(10) Pub. No.: US 2015/0011929 A1
(43) Pub. Date: Jan. 8, 2015

(54) VIBRATING CATHETER LUER ACCESSORY

(71) Applicant: THE JOHNS HOPKINS UNIVERSITY, Baltimore, MD (US)

(72) Inventors: Shuja Dawood, Baltimore, MD (US); Adam Clark, Baltimore, MD (US); Nathaniel Moller, Baltimore, MD (US); Luis Soenksen, Baltimore, MD (US); Soumyadipta Acharya, Baltimore, MD (US); Steven Hsu, Baltimore, MD (US); Clifford Weiss, Annapolis, MD (US)

(21) Appl. No.: 14/379,025

(22) PCT Filed: Feb. 18, 2013
(86) PCT No.: PCT/US2013/026581

§ 371 Date: Aug. 15, 2014

Related U.S. Application Data
(60) Provisional application No. 61/599,504, filed on Feb. 16, 2012.

(51) Int. Cl. A61M 25/00; A61B 19/00

(52) U.S. Cl. CPC A61M 25/0017; A61B 19/34; A61M 2025/0019; A61M 39/16
USPC 604/22

(57) ABSTRACT

A device and method of the present invention provides application of low-energy acoustic waves to indwelling surfaces of a catheter in order to remove and prevent microbial biofilm formation. The low-energy acoustic waves are generated by an electrically activated piezo element. The device can take the form of a luer connector configured to couple to the hub of the indwelling catheter or can take the form of a catheter insert. The characteristics of the acoustic waves can be varied in order to inhibit bacterial adhesion to the indwelling surfaces of the catheter. Moreover, the characteristics of the acoustic waves must also be in a range so as to not induce bacterial adhesion to the indwelling catheter surfaces.

### CROSS REFERENCE TO RELATED APPLICATIONS

This application claims the benefit of U.S. Provisional Patent Application No. 61/599,504 filed Feb. 16, 2012, which is incorporated by reference herein, in its entirety.

### FIELD OF THE INVENTION

The present invention relates generally to venous access. More particularly the present invention relates to a device for preventing biofilm from forming on an indwelling catheter.

### BACKGROUND OF THE INVENTION

Biofilm is a structured community of bacteria, proteins, red blood cells, platelets, and many other types of cells that are adherent to an inert or living surface. The biofilm provides a sticky surface for continued buildup of cells. This buildup eventually occludes the catheter and limits its functionality. This is detrimental to patients, as central venous catheters (CVCs) are used for clinical applications such as administration of chemotherapy drugs and hemodialysis. Studies have shown that 28% of catheters experience some sort of dysfunction within 7 days after implantation. In cases where occlusion occurs, physicians will replace the catheter or attempt to salvage it by physically removing the source of occlusion. However, the cost to replace a catheter is up to $1500, and the cost to salvage is up to $350. Expenses quickly add up if either of these procedures must be repeated.

Another, perhaps more detrimental, problem caused by biofilms is catheter-related blood stream infections (CRBSIs). Biofilm contains bacteria that are typically responsible for CRBSIs. This includes Staphylococcus aureus, Enterococcus faecalis, Escheria coli, Psedumonas aeruginosa, and Candida albicans. The biofilm layer protects the bacteria from antibiotics and other forms of treatment. It is estimated that 280,000 CRBSIs occur every year, of which 90% (250,000) are attributed to CVCs. Approximately 12-25% of these cases are fatal. The cost per case of CRBSI is up to $56,000 resulting in a total annual health care cost of $2.3 billion. To exacerbate the situation, since 2008, Centers for Medicare and Medicare Services (CMS) does not reimburse for hospital acquired CRBSIs. The average loss for a hospital per case is $27,000.

Overall, 76% of CVCs will develop complications (occlusions or CRBSI) related to biofilm development. These complications affect patient treatment and increase morbidity and mortality.

It would therefore be advantageous to provide a device and method for preventing biofilm formation on CVCs to decrease the risk of patient morbidity and mortality and significantly decrease patient management costs.

### SUMMARY

The foregoing needs are met, to a great extent, by the present invention, wherein in one aspect a device for delivering energy to from a surface of an indwelling catheter includes a connector configured to attach to the surface of the indwelling catheter. The device includes a source of vibrations configured to transmit energy to the surface of the indwelling catheter. Additionally, the source of vibrations is coupled to the connector, such that the vibrations are delivered to the surface of the indwelling catheter.

In accordance with an aspect of the present invention, the vibrations are mechanical and/or acoustic. The source of vibrations is configured to transmit high and low energy vibrations to the surface of the indwelling catheter, and the source of vibrations can take the form of a piezoelectric crystal. The piezoelectric crystal is configured to provide vibrations and is not controlled by a processor. The source of vibrations is configured to provide vibrations in a range between approximately 0.05 to 0.20 mW/cm² and is further configured to provide vibrations below 0.35 mW/cm². The source of vibrations is powered with a battery.

In accordance with another aspect of the present invention, a device delivering energy to a surface of an indwelling catheter includes a connector having a proximal end and a distal end. The distal end of the connector is configured to couple to a luer connector of an indwelling catheter. A sheath is coupled to the distal end of the connector. The sheath includes an outer surface defining an inner lumen, and the sheath is configured to be disposable within a lumen of the indwelling catheter. A source of vibrations is coupled to the connector and configured to transmit vibrations along a length of the sheath, such that the vibrations are also transmitted to the surface of the indwelling catheter. The connector can be configured for infusions and draws. The connector is in fluid communication with the inner lumen of the sheath and the inner lumen of the sheath is in fluid communication with the lumen of the indwelling catheter. Additionally, the sheath has a stiffness configured to promote wave propagation and minimize a dampening effect.

In accordance with another aspect of the present invention, a device for delivering energy to a surface of an indwelling catheter includes a male luer connector having a proximal end and a distal end, and a female luer connector having a proximal end and a distal end. A source of vibrations is configured to transmit energy to the surface of the indwelling catheter. The source of vibrations is disposed between a distal end of the male luer connector and a proximal end of the female luer connector. The male luer connector and the female luer connector define a lumen between the proximal end of the male luer connector and the female luer connector and a tube of the indwelling catheter is disposed within said lumen, such that the male and female luer connectors are in fluid communication with each other and the indwelling catheter.

In accordance with yet another aspect of the present invention, the device includes a core material around which the source of vibrations is disposed. The core material is coupled to the distal end of the male luer connector.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIGS. 1A and 1B illustrate a schematic diagram of a mechanism of piezo vibration and mechanical stimulation of surfaces, according to an embodiment of the present invention.

FIGS. 2A-2C illustrate a piezo activator and vibration clip for an indwelling catheter, according to an embodiment of the present invention.

FIGS. 3A and 3B illustrate an internal sheath piezo vibration device for an indwelling catheter, according to an embodiment of the present invention.

FIGS. 4A-4D illustrate a piezoelectric add-on component for standard male and female luer connectors, according to an embodiment of the present invention.

FIG. 5 illustrates an electrical component and battery housing according to an embodiment of the present invention.

FIGS. 6A and 6B illustrate perspective views of a control board for controlling the source of acoustic vibrations, according to an embodiment of the present invention.

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

A device and method of the present invention provides application of low-energy acoustic waves to indwelling surfaces of a catheter in order to remove and prevent microbial biofilm formation. The low-energy acoustic waves are generated by an electrically activated piezo element. The device can take the form of a luer connector configured to couple to the hub of the indwelling catheter or can take the form of a catheter insert. The characteristics of the acoustic waves can be varied in order to inhibit bacterial adhesion to the indwelling surfaces of the catheter. Moreover, the characteristics of the acoustic waves must also be in a range so as to not induce bacterial adhesion to the indwelling catheter surfaces.

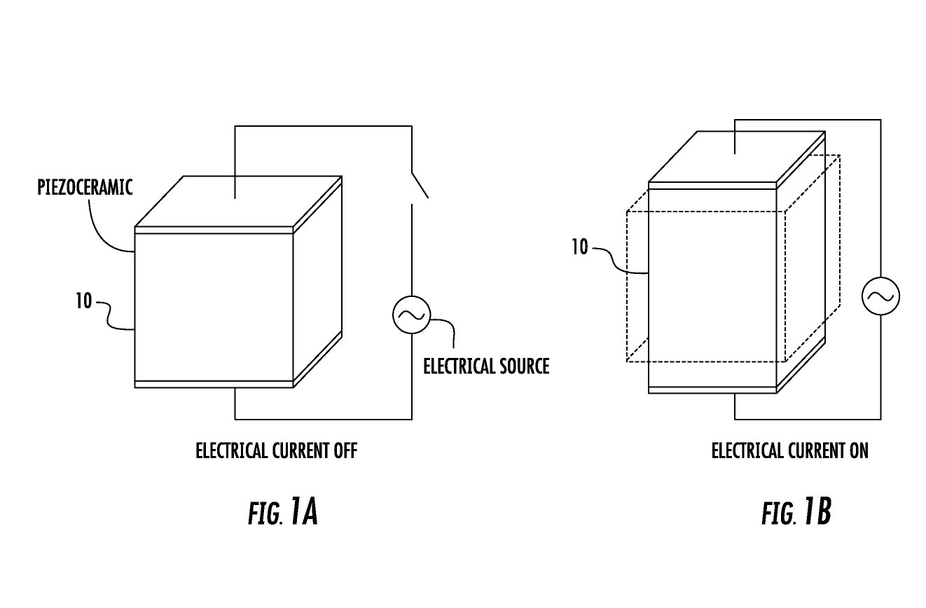

FIGS. 1A and 1B illustrate a schematic diagram of a mechanism of piezo vibration and mechanical stimulation of surfaces, according to an embodiment of the present invention. More particularly, FIG. 1A illustrates the mechanism of piezo vibration and mechanical stimulation of surfaces 10 with the electrical current off, and FIG. 1B illustrates the mechanism of piezo vibration and mechanical stimulation of surfaces 10 with the electrical current on. The mechanism 10 preferably is configured to apply surface waves that propagate through all surfaces of the indwelling catheter. The mechanism 10 is also configured to produce waves having a nanometric amplitude with a power intensity below 1.1 mW/cm². Frequencies of the waves produced by the mechanism 10 should range from 100 to 300 kHz. Preferably, an acoustic energy range between 0.05-0.20 mW/cm² is used to inhibit E. coli adhesion to red blood cells. It should be noted that increased acoustic energy or acoustic amplification above 0.35 mW/cm² has shown to induce strong bacteria-mediated adhesion of red blood cells and enhanced aggregation on the indwelling surfaces. Therefore, it is important that the mechanism 10 be configured to produced and maintain acoustic energy within the prescribed range. The mechanism 10 preferably is also configured to enable optimal propagation and preventive effects, because skin and tissue can have a dampening effect on longitudinal and transversal dispersion of the waves.

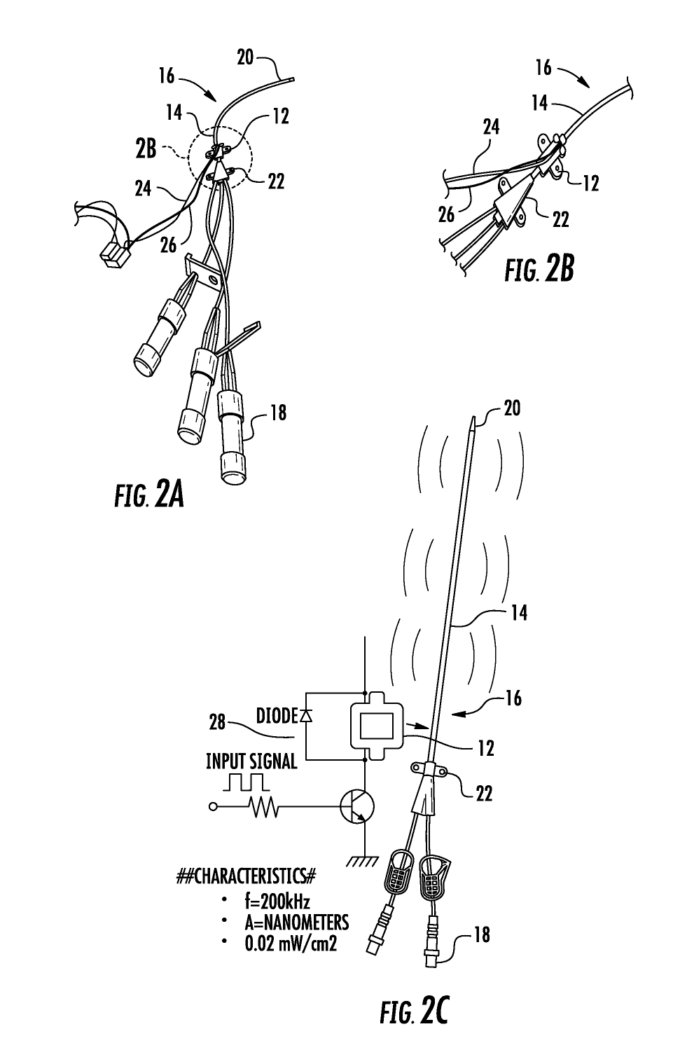

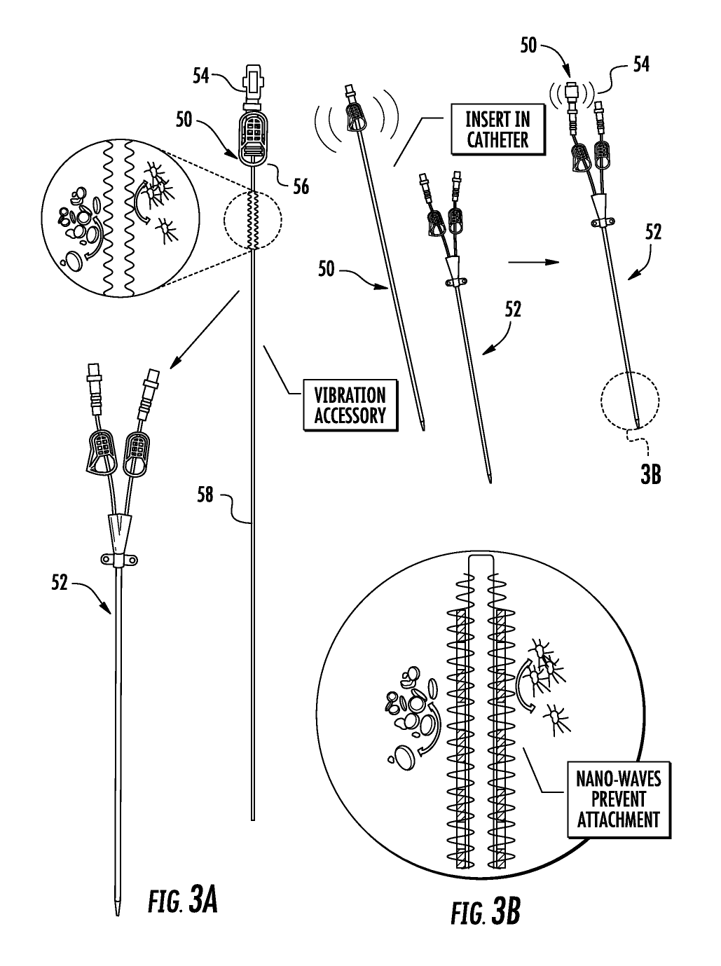

FIGS. 2A-2C illustrate a piezo activator and vibration clip for an indwelling catheter, according to an embodiment of the present invention. FIGS. 2A and 2B illustrate a piezo activator and vibration clip 12 disposed on an outer surface 14 of an indwelling catheter 16. The indwelling catheter 16 has a proximal end 18 and a distal end 20. The piezo activator and vibration clip 12 are shown in FIG. 2A as being positioned just distal to a hub 22 of the indwelling catheter 16. However, the piezo activator and vibration clip 12 can be positioned in any location on the indwelling catheter 16 to facilitate removal and prevention of biofilm. Also, the piezo activator and vibration clip 12 includes a first wire 24 and a second wire 26 to provide electrical current to the clip 12. The wires 24 and 26 can be connected to a battery (not shown) or any other source of electrical current known to or conceivable by one of skill in the art to power the clip 12. The physical attachment of the vibrating material to the catheter's external surface creates the interface for the generation of the surface acoustic waves. More particularly, a 9-volt battery is proposed to generate a power intensity of 0.02 mW/cm² in a 5×5×6.6 mm prismatic piezo ceramic block with a resonant frequency of 200 kHz.

FIG. 2C illustrates an exploded view of the indwelling catheter and piezo activator and vibration clip of FIGS. 2A and 2B, according to an embodiment of the present invention. The piezo activator and vibration clip 12 can take the form of an oscillating circuit 28 with voltage amplification that attaches to an outer surface 14 of the indwelling catheter 16. When activated, the piezo activator and vibration clip 12 creates surface acoustic waves down the length of the indwelling catheter 16.

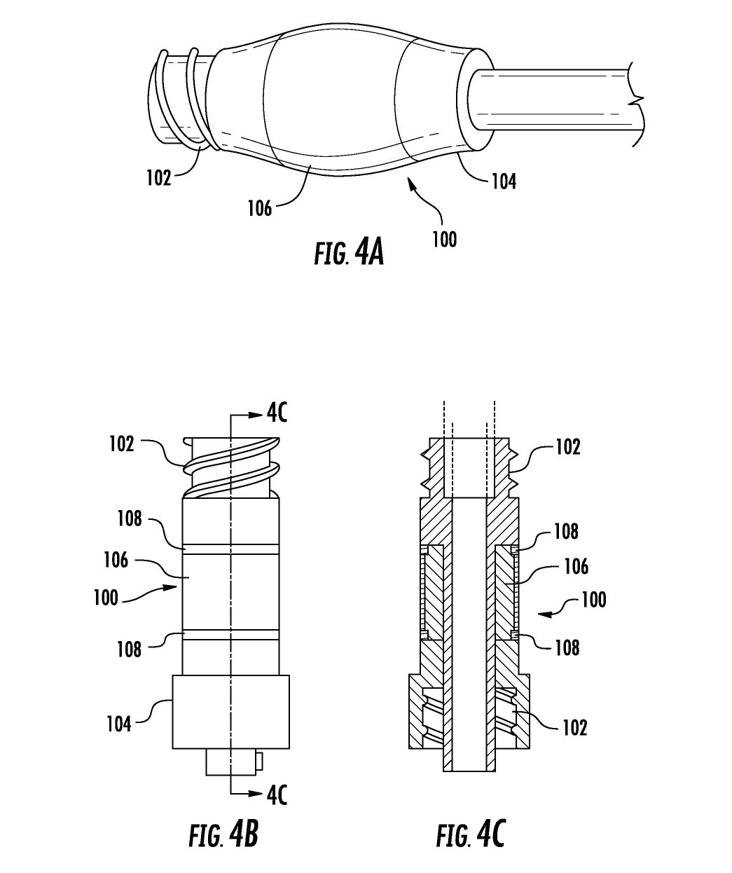

FIGS. 3A and 3B illustrate an internal sheath piezo vibration device for an indwelling catheter, according to an embodiment of the present invention. More particularly, FIG. 3A illustrates an exploded view of an internal sheath piezo vibration device 50 for insertion into an indwelling catheter 52, and FIG. 3B illustrates the internal sheath piezo vibration device 50 inserted into the indwelling catheter. The internal sheath piezo device 50 can be inserted into any of the lumens of the indwelling catheter 52. A connector 54 is positioned on a proximal end 56 of the internal sheath piezo device 50. The connector 54 is coupled to a sheath-like tube 58 that can be inserted into one of the lumens of the indwelling catheter 52. The connector 54 can allow for standard infusions and/or draws. Additionally, the connector has standard luer connections and is in fluid communication with a lumen of the sheath-like tube 58.

Further as illustrated in FIGS. 3A and 3B, the internal sheath device 50 can be configured to be insertable and removable from the lumen of the indwelling catheter or can be permanently placed in the lumen of the indwelling catheter. Preferably, the sheath-like tube 58 has an external diameter that is approximately equal to an internal diameter of the accessed indwelling catheter lumen. It is also preferable that the sheath-like tube have a minimal wall thickness, so as to not greatly decrease the internal diameter of the lumen of the indwelling catheter 52. The sheath-like tube 58 can extend along the entire length of the lumen of the indwelling catheter, or can also be of a length shorter than that of the entire length of the lumen of the indwelling catheter, so long as vibrations are delivered along the entire length of the indwelling catheter. Once placed the sheath-like tube 58 secured tightly against the internal wall of the catheter 52, fixing both systems. The sheath-like tube 58 should be formed from a material having an appropriate stiffness to promote wave propagation with minimal dampening effect, to enable homogenous propagation of the low intensity mechanical vibrations throughout the catheter. The internal sheath device 50 can also add power injectable capabilities to catheters lacking that feature. Preferably, a miniaturized, monofrequency/oscillating RC circuit with voltage amplification will be used to integrate vibration and power source in a lightweight connector. Alternately, a piezo element can be placed in the connector and a snap electrical connection could be used in conjunction with a separated circuit patch containing the other elements of the system.

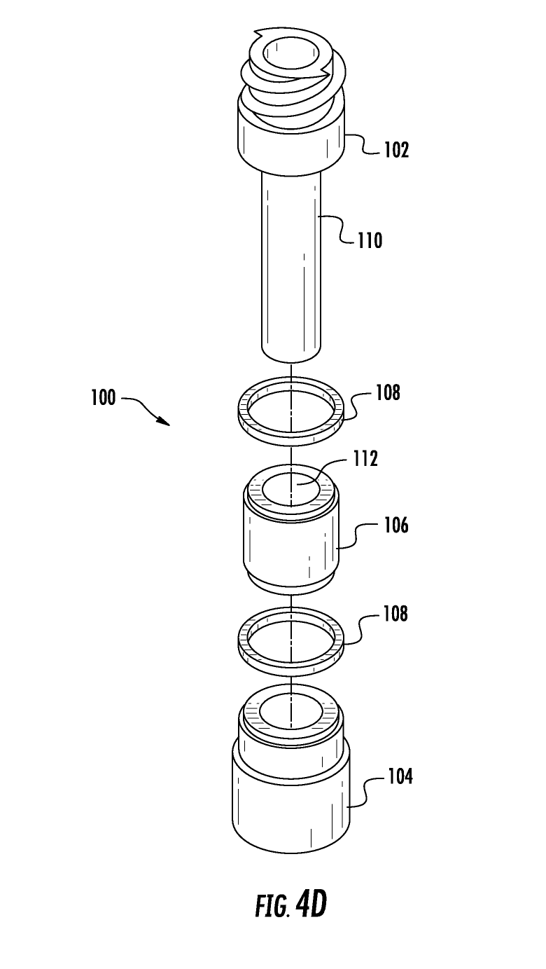

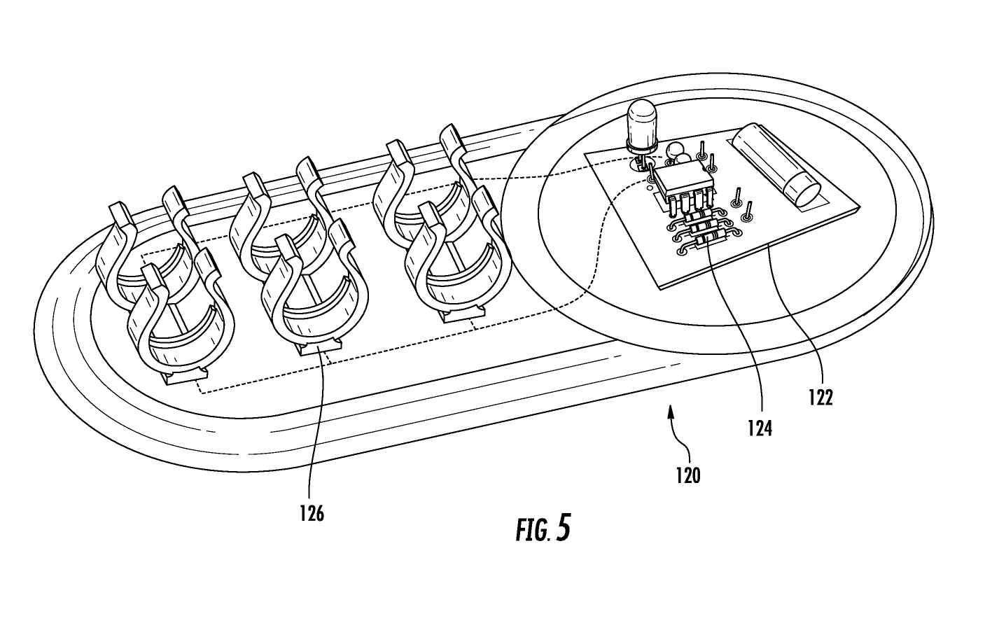

FIGS. 4A-4D illustrate a piezoelectric add-on component for standard male and female luer connectors, according to an embodiment of the present invention. More particularly, FIG. 4A illustrates the piezoelectric add on component 100 coupled to a standard catheter connection hub, according to an embodiment of the present invention. FIG. 4B illustrates a side view of the piezoelectric add on component 100 illustrated in FIG. 4A, and FIG. 4C illustrates a sectional view of the component 100 illustrated in FIG. 4B. Additionally, FIG. 4D illustrates an exploded view of the component 100. The component 100 includes a standard male luer connector 102 and a standard female luer connector 104. A piezoelectric ring actuator 106 is sandwiched between the male luer connector 102 and the female luer connector 104. Metallic rings 108 can also be positioned between the piezoelectric actuator ring 106 and the male and female luer connectors 102, 104. The component 100 is further configured to couple to a standard indwelling catheter tube, such that it can provide acoustic vibration to the surfaces of the indwelling catheter, to prevent biofilm buildup. The male luer connector 102 can also include core material 110 that sits in an inner lumen 112 of the piezoelectric ring actuator 106. the core material 110 should also be configured such that it maintains the fluid communication between the male luer connector 102 and the female luer connector 104.

FIG. 5 illustrates an electrical component and battery housing according to an embodiment of the present invention. The electrical component and battery housing 120 includes a hidden astable oscillating circuit driver and battery 122. The housing 120 also includes electrical connections 124 for the activation of the acoustic vibration source coupled to the indwelling catheter, such as a piezo device. The electrical connections 124 can take the form of invisible connections, or any other form of suitable connection known to one of skill in the art. The housing 120 can also include snap connections 126 for docking devices such as the add-on component discussed above with respect to FIGS. 4A-4D. The snap connections 126 can be configured to activate the acoustic vibration actuator in the devices by completing a circuit within the devices. Alternately, the electrical component and battery housing can be incorporated directly into the design of any of the embodiments described above. The battery contained within the electrical component and battery housing 120 can take the form of an off-the shelf battery known to one of skill in the art, or any custom battery known to or conceivable by one of skill in the art, and appropriate for powering the invention. The housing 120 can be worn on a necklace or adhered to the patient's skin. The housing 120 may also be configured such that it is reusable. Alternately, the housing 120 can be configured such that it is incorporated within the main body of the device.

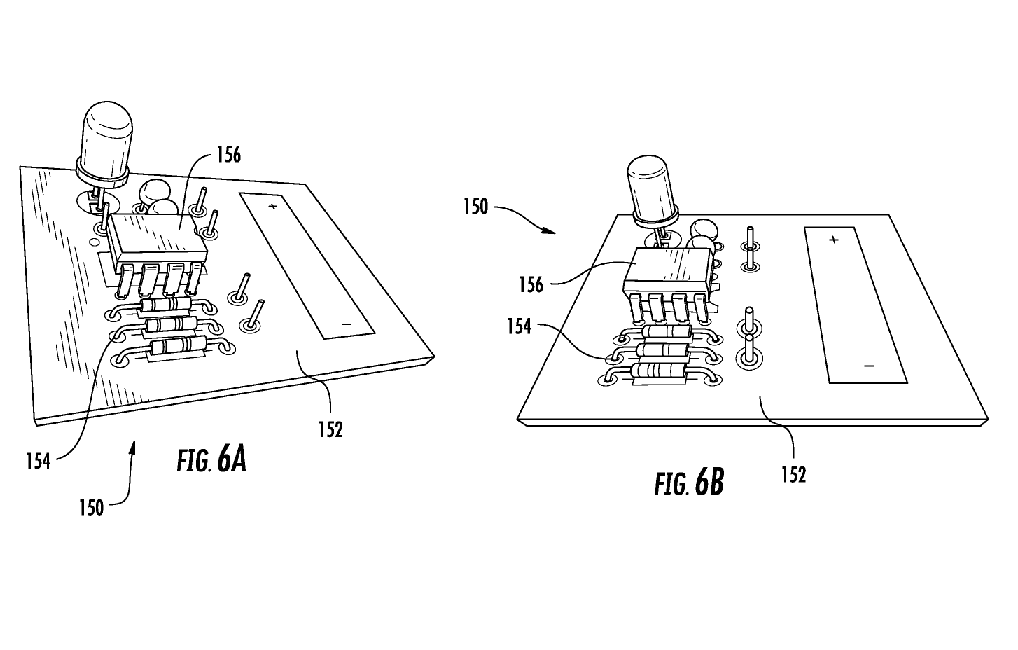

FIGS. 6A and 6B illustrate perspective views of a control board for controlling the source of acoustic vibrations, according to an embodiment of the present invention. The control board 150 is configured to control a duty cycle of the vibrations without the use of a micro controller. A duty cycle can be used in order to discourage biofilm formation. Additionally, use of the duty cycle can extend battery life. The control board 150 can take the form of a printed circuit board 152, timer 154, and chips 156.

Any of the proposed embodiments of the device can incorporate a needleless connection system. Preferably, the invention includes an elastic septum allowing luer access but prohibiting needle use. A pre-pierced elastomer can be used to ensure a normally closed state. When interfacing with a needleless connector, the compression forces would displace the septum and open the pre-pierced channel, opening the fluid pathway. The spring forces applied to the normally closed septum will be achieved via septum design, material selection and/or additional spring components. This connection can also be designed for compatibility with pressure injection pressures and flow rates such as for example 350 psi and 10 mL/sec, respectively.

Further, with respect to the embodiments of the present invention, any housing components for the invention can be injection molded as a single piece. For instance, a housing can be molded to encapsulate the inner components such as a piezoelectric actuator. The housing can further be formed such that the inner components can be inserted via a linear or stacking process with a press or snap fit to secure the components in place. Alternately, the housing can be molded in one piece which van be rotated or folded in half to close around the inner components. Once the two halves are rotated together, a feature will interface, such as a slot and lever or snap fitting that will make opening the device impossible without compromising its functionality. Any housing components can be manufactured from commonly used medical device materials known to one of skill in the art, such as ABS.

Any luer connectors used with the system can take the form of standard male and female luer connectors in compliance with ISO 594-1/2. The housing shell can be broken into three main components as illustrated in FIGS. 4A-4D above. Alternately, the housing can be molded as one single piece, incorporating the male and female luers. Additionally, the piezoelectric component can be bonded to the housing or connectors using an interference fit or medical grade bonding agent such as cyclohexanone. In each of the embodiments, a core component can be fixed to the piezoelectric material and can also interface with an inner lumen of the indwelling catheter. The core component can be connected to the piezoelectric material using an adhesive or interference fit.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A device for delivering energy to a surface of an indwelling catheter comprising:
a connector configured to attach to the surface of the indwelling catheter;
a source of vibrations configured to transmit the energy to the surface of the indwelling catheter; and
wherein said source of vibrations is coupled to the connector, such that the energy is delivered to the surface of the indwelling catheter.

2. The device of claim 1 wherein the vibrations are one selected from a group consisting of mechanical and acoustic.

3. The device of claim 1 wherein the source of vibrations is configured to transmit high and low energy vibrations to the surface of the indwelling catheter.

4. The device of claim 1 wherein the source of vibrations is a piezoelectric crystal.

5. The device of claim 4 wherein the piezoelectric crystal is configured to provide vibrations and is not controlled by a processor.

6. The device of claim 1 wherein the source of vibrations is configured to provide vibrations in a range between approximately 0.05 to 0.20 mW/cm².

7. The device of claim 1 wherein the source of vibrations is configured to provide vibrations below 0.35 mW/cm².

8. The device of claim 1 wherein the source of vibrations is powered with a battery.

9. A device for delivering energy to a surface of an indwelling catheter comprising:
a connector having a proximal end and a distal end wherein said distal end is configured to couple to a luer connector of an indwelling catheter;
a sheath coupled to the distal end of the connector, wherein said sheath includes an outer surface defining an inner lumen, and wherein said sheath is configured to be disposable within a lumen of the indwelling catheter; and
a source of vibrations coupled to the connector and configured to transmit vibrations along a length of the sheath such that the vibrations are also transmitted to the surface of the indwelling catheter.

10. The device of claim 9 wherein the connector is configured for infusions and draws.

11. The device of claim 9 wherein the connector is in fluid communication with the inner lumen of the sheath and the inner lumen of the sheath is in fluid communication with the lumen of the indwelling catheter.

12. The device of claim 9 wherein the sheath comprises a stiffness configured to promote wave propagation and minimize a dampening effect.

13. The device of claim 9 wherein the vibrations are one selected from a group consisting of mechanical and acoustic.

14. The device of claim 9 wherein the source of vibrations is configured to transmit high and low energy vibrations to the surface of the indwelling catheter.

15. The device of claim 9 wherein the source of vibrations is a piezoelectric crystal.

16. The device of claim 15 wherein the piezoelectric crystal is configured to provide vibrations and is not controlled by a processor.

17. The device of claim 9 wherein the source of vibrations is configured to provide vibrations in a range between approximately 0.05 to 0.20 mW/cm².

18. The device of claim 9 wherein the source of vibrations is configured to provide vibrations below 0.35 mW/cm².

19. The device of claim 9 wherein the source of vibrations is powered with a battery.

20. A device for delivering energy to a surface of an indwelling catheter comprising:
a male luer connector having a proximal end and a distal end;
a female luer connector having a proximal end and a distal end;
a source of vibrations configured to transmit the energy to the surface of the indwelling catheter, wherein said source of vibrations is disposed between a distal end of the male luer connector and a proximal end of the female luer connector; and
wherein the male luer connector and the female luer connector define a lumen between the proximal end of the male luer connector and the female luer connector, such that the male and female luer connectors are in fluid communication with each other and the indwelling catheter.

21. The device of claim 20 further comprising a core material around which the source of vibrations is disposed.

22. (canceled)

23. The device of claim 21 wherein the core material is coupled to the distal end of the male luer connector.

24. The device of claim 20 wherein the vibrations are one selected from a group consisting of mechanical and acoustic.

25. The device of claim 20 wherein the source of vibrations is configured to transmit high and low energy vibrations to the surface of the indwelling catheter.

26. The device of claim 20 wherein the source of vibrations is a piezoelectric crystal.

27. The device of claim 26 wherein the piezoelectric crystal is configured to provide vibrations and is not controlled by a processor.

28. The device of claim 20 wherein the source of vibrations is configured to provide vibrations in a range between approximately 0.05 to 0.20 mW/cm².

29. The device of claim 20 wherein the source of vibrations is configured to provide vibrations below 0.35 mW/cm².

30. The device of claim 20 wherein the source of vibrations is powered with a battery.
